(19) United States
(12) Patent Application Publication
(10) Pub. No.: US 2005/0278012 A1
(43) Pub. Date: Dec. 15, 2005

(54) Protected stent delivery system and packaging

(75) Inventor: Carlos Vonderwalde, Richmond (CA)

Correspondence Address: Martin D. MOYNIHAN;c/o Anthony Castorina, Suite 207, 2001 Jefferson Davis Highway, Arlington, VA, 22202, US

(73) Assignee: Design & Performance - Cyprus Limited

(21) Appl. No.: 10/865,379

(22) Filed: Jun. 10, 2004

(51) Int. Cl. A61F 2/06

(52) U.S. Cl. 623/1.11; 606/108; 206/438

(57) ABSTRACT

The present invention is directed to sterile packaging systems for medical devices or components where the medical device or component (e.g. a stent) is covered at least in part with a protective sheath that is configured to maintain a body of protective fluid about the covered portion of the device or component. The protective sheath preferably has one or more ports to add fluid to or withdraw fluid from the interior of the protective sheath. The partially protected article is preferably maintained in a sterile condition within a sealed container formed at least in part of a material permeable to sterilizing gas so the protected article within the interior of the sealed container may be subjected to an atmosphere of sterilizing gas which permeates into the interior of the sealed container and sterilizes the partially protected article within the sealed container. In one embodiment a protected stent delivery system is disposed in a covered but unsealed tray which in turn is sealed within a pouch formed at least in part of a material which is permeable to the sterilizing gas.

### BACKGROUND OF THE INVENTION

The present invention relates to the delivery of expandable endoprosthesis devices, generally called stents, which are adapted to be implanted into a patient's body lumen, such as a blood vessel, to maintain the patency thereof. Stents are particularly useful in the treatment and repair of blood vessels after or during percutaneous transluminal coronary angioplasty (PTCA), percutaneous transluminal angioplasty (PTA), or atherectomy and reduce the possibility of restenosis. They may also be employed to treat vulnerable plaque.

Stents are cylindrically shaped, usually metallic tubular devices which function to hold open and sometimes expand a segment of a blood vessel or other body lumen, such as coronary artery. Stents are usually delivered in a compressed condition to the target site and then deployed at that location into an expanded condition to support the vessel and help maintain it in an open position. They are also suitable for use to support and hold back a dissected arterial lining after an angioplasty procedure to avoid occlusion of the arterial passageway.

A variety of stent designs have been used. One of the difficulties encountered in prior art stents involve maintaining the radial rigidity needed to hold open a body lumen, while at the same time maintaining the longitudinal flexibility of the stent to facilitate its delivery and accommodate the often tortuous path of the body lumen.

PTCA is a well established minimally invasive procedure for the treatment of heart disease, wherein a balloon catheter is advanced within the patient's vasculature until the balloon on the catheter is disposed within the arterial blockage and the balloon is inflated to expand the blockage to thereby increase the blood flow therethrough. In a typical PTCA procedure, a guiding catheter is first percutaneously inserted into the patient's cardiovascular system either through the brachial or the femoral arteries, and is advanced until the distal tip of the guiding catheter is seated within the ostium of the desired coronary artery. A balloon dilatation catheter is then advanced out of the guiding catheter into a patient's coronary artery through the inner lumen of the guiding catheter, until the balloon at the distal portion of the catheter is disposed within the desired region of the patient's artery. The balloon is inflated and deflated one or more times as required to re-open the arterial passageway and thereby permit blood flow volume to increase once the catheter is removed.

Most angioplasty procedures today involve placement of a stent at the site to minimize restenosis and generally provide scaffolding support to the arterial region. Typically, the stent is delivered with the balloon expansion to dilate the stenosis. However, the stent may be delivered after the angioplasty procedure is completed with another balloon catheter which is similar to the balloon catheter used for the angioplasty procedure.

There are generally two types of catheters used in PTCA/stent delivery procedures, namely the rapid exchange type balloon catheters and the over-the-wire type balloon catheters. However, by far, most stents are delivered with rapid exchange type delivery catheters.

A rapid exchange type balloon catheter has a relatively short guide wire receiving lumen extending through a distal portion of the catheter with one guide wire port at the distal end of the catheter and another guide wire port spaced about 5 to about 50, usually about 10 to about 40 cm, from the distal end. These catheters allow for the rapid exchange of the catheter without the need for an exchange wire or adding a guide wire extension to an in-place guide wire. Over-the-wire balloon catheters have guide wire lumens which extend the entire length of the catheter and require guide wire extensions or exchange wires to exchange the catheter.

Key features for effective stent delivery include delivery of the stent through tortuous anatomy without damage to the stent or displacement of the stent from the balloon. This requires that the catheter be highly responsive to the controlled advancement of the balloon catheter with optimal pushability from the proximal shaft section, and at the same time, retaining overall optimal flexibility for advancement within the tortuous anatomy of the patient's vascular structures.

The present invention relates generally to sterilized medical devices and packaging for sterilized medical devices. In particular, the present invention relates to the packaging of sterilized environmentally sensitive medical devices, such as stents covered with biological materials such as pericardial tissue and drug-eluting stents.

Most, if not all, disposable medical devices which are designed to come into contact with patients, require sterilization prior to or concurrent with packaging. The packaging, which may be in the form of a pouch, bag, tube, box, or other sealed container, will maintain the sterility of the product until it is withdrawn from the packaging for use. Typically, sterile packaging of medical devices relies on use of spun high-density polyethylene (HDPE) pouch or other container which are used to enclose the medical device or other product. The device may be placed in the package prior to sterilization. Sterilization is then effected by placing the package in an environment including a sterilant gas, such as ethylene oxide (EtO). The spun HDPE material, such as Tyvek®, which is available from Medical Packaging Division of E. I DuPont de Nemours and Company (DuPont), permits passage of the small molecule sterilant gas to the interior of the container or pouch while remaining a barrier to bacteria, viruses, and other larger substances which might compromise sterility. The packaging material also acts as a barrier to water and other fluids which might detrimentally affect the sterilized contents.

While this packaging system has been widely used for conventional medical devices, such as catheters, stents, surgical instruments, probes, and the like, it is frequently not suitable for medical devices which have jackets, coatings or components which are subject to oxygen or moisture degradation. For example, in U.S. Pat. No. 6,699,277, which issued Mar. 2, 2004, a stent is described having a jacket formed of heterologous tissue such as pericardial tissue, e.g. bovine, porcine or equine pericardial tissue. The pericardial tissue of these jackets can degrade without protection. Similarly, the use of drug-eluting stents (DES) is promising to revolutionize interventional cardiology. The DES are coated with plastic coating having an incorporated drug such a paclitaxel or rapamycin. These DES are made at a central fabrication facility and must be distributed to the end user in sterile packaging. While at least some of the drugs presently contemplated for use in DES will remain stable during ethylene oxide sterilization, exposure to oxygen during lengthy distribution and storage periods can adversely affect the drug.

Thus, there is a need for protecting jacketed and coated stents and the delivery systems therefore. The present invention satisfies these and other needs.

### SUMMARY OF THE INVENTION

The present invention is directed to a protected delivery system for a medical device or component, such as a jacketed stent having a decomposable or degradable jacket or a drug eluting stent, where the stent coating or drug incorporated into the stent coating is degradable in the environment in which they are stored. The present invention is also useful with pre-crimped covered or coated stents which are not compatible with standard sterilization procedures and which need to be protected and/or kept moist.

The protected delivery system embodying features of the invention preferably includes a delivery catheter which has an elongated shaft, a distal extremity on the elongated shaft, and a stent expanding member on the distal extremity of the catheter. A jacketed or coated stent is mounted on the stent expanding member on the distal extremity of the catheter. A sealable container or sheath is disposed about the distal extremity of the catheter having an interior receiving the expandable member and the stent mounted thereon. The sheath or container may then be sealed. The container or sheath is configured to maintain a body of protective fluid about the mounted stent to ensure that the device is ready to be used by the physician when the package is open with little or no preparation. The protective fluid may be a liquid, a slurry, a gel or a gas. The sheath is preferably provided with one or more sealable ports to add or withdraw fluid from the interior of the sheath. For example, the protective fluid may be withdrawn and the distal portion of the catheter and the mounted stent with a jacket or coating may be rinsed with sterile water or saline prior to introduction into the patient. The distal extremity of the catheter and the mounted stent are sterilized prior to placement of the protective fluid within the container or sheath and the sealing thereof.

The type of protective fluid maintained within the interior of the sheath will depend upon the protection needed or desired. For example, in the case of a stent with a jacket formed of pericardial tissue, the protective fluid might be glutaraldehyde. In other cases, the fluid may be sterile water or sterile saline. Gaseous fluids such as inert or otherwise non-reactive (to the coating or jacket or any drugs contained therein) gas such as Nitrogen may also be employed. The protective fluid may also contain a drug or diagnostic agent in order to impregnate the stent or the stent cover or to maintain a proper drug concentration.

The protected delivery system may be stored or transported as is and further sterilized on site. However, the protected delivery system is preferably part of a sterilized packaging. system. For example, the packaging system may include a sealable container configured to receive the protected delivery system. The container may be formed at least in part of a material, e.g. a spun high density polyethylene such as Tyvec which is permeable to sterilizing gas such as ethylene oxide (EtO). The EtO may be mixed with up to 80% of otherwise non reactive gases. Other gaseous sterilizing agents include Aprolene and hydrogen peroxide.

In one packaging system embodying features of the invention, the container is a tray sealed with a cover which is configured to receive the protected delivery system. The sealed covered tray is formed in part of a material as described above which is permeable to the sterilizing gas but which impermeable to bacteria, viruses, water and other detrimental materials. The contents of the covered tray, the protected delivery system, may be sterilized by maintaining a sterilizing gas in contact with at least the portion of the covered tray which is formed of the permeable material. In this embodiment, the tray and cover are preferably sealed prior to sterilization.

In another packaging system embodying features of the invention, an unsealed covered tray is placed in a pouch or envelope that is formed at least in part of a material which is permeable to sterilizing gas and the pouch or envelope then sealed. The pouch or envelope is then subjected to a sterilizing gas atmosphere as described above to sterilize the covered tray and its contents.

A suitable material which is permeable to sterilizing gas and which generally has the requisite mechanical and other properties for sterile packaging for medical products is a spun high density polyethylene such as Vytek® (2FS, 1059B and 1073B) which is available from DuPont.

The catheter utilized in the protected stent delivery system may be a conventional stent delivery catheter (over-the-wire or rapid exchange) or may be of the design described in co-pending application Ser. No. 10/735,548, filed on Dec. 12, 2003, which has been assigned to the present assignee. The delivery catheter for percutaneous deployment to the desired intracorporeal location generally has an elongated shaft with an inflation lumen and a guide wire lumen extending therein and an inflatable member or balloon which is disposed about a distal shaft section. A inflatable member is configured to expand a stent mounted on the exterior thereof.

The stent is mounted on the working section of the balloon for delivery, usually by crimping, and is generally the same length or a little shorter than the working length of the balloon. See for example U.S. Pat. No. 6,605,107.

The interior of the covered tray may be evacuated to aspirate sterilizing gas surrounding the pouch or covered tray to facilitate the passage of sterilizing gas through the permeable portion of the covered tray or pouch. The pressure of the sterilizing gas is maintained within the container interior at suitable levels for effective sterilization. The dwell time of the sterilizing gas within the container interior depends upon the nature and pressure of the sterilizing agent. After sterilization, the interior can be partially or totally evacuated of oxygen or an oxygen containing gas and replaced totally or partially with a non-reactive gas such as nitrogen. The interior may be flushed or purged with a non reactive gas one or more times in order to reduce the level of sterilizing gas within the interior.

In some cases, it may be further desirable to provide desiccant materials within the sealed covered tray or within the pouch in order to sequester any moisture which may remain within the pouch after final sealing. Alternatively, a small canister of the desiccant material may be provided within the enclosure, similar to the desiccant placed in conventional pharmaceutical packaging.

The invention provides a simple, inexpensive protection and packaging of medical devices and components which are formed of or are coated or covered with degradable or labile materials. These and other advantages of the invention will become more apparent from the following detailed description of the invention when taken in conjunction with the accompanying exemplary drawings.

### BRIEF DESCRIPTION OF THE DRAWINGS

FIG. 1 is an elevational view of a protected stent delivery system, partially in section, which embodies features of the invention.

FIG. 2a is an exploded perspective view of a tray, the protected stent delivery system shown in FIG. 1 and a protective cover.

FIG. 2b is a partially exploded perspective view with the protected stent delivery system disposed within the recess of the tray and the cover.

FIG. 2c is a perspective view of the tray, the protected stent delivery system and the cover completely closed.

FIG. 2d is a perspective view of the closed covered tray being inserted into a sterilizable pouch.

FIG. 2e is an elevational view, in section of the covered tray in the sealed pouch.

FIG. 3 is a transverse cross sectional view of the covered tray shown in FIG. 2c, taken along lines 3-3.

FIG. 4 is a transverse cross sectional view of the covered tray shown in FIG. 2c, taken along lines 4-4.

FIG. 5a is an exploded, perspective view of a covered stent delivery system and packaging therefore.

FIG. 5b is an exploded, perspective view of a covered stent delivery system and packaging therefore shown in FIG. 5a with the stent delivery system disposed within the recess of the packaging tray.

FIG. 5c is a perspective view of a covered stent delivery system disposed within packaging therefore.

### DETAILED DESCRIPTION OF EMBODIMENTS OF THE INVENTION

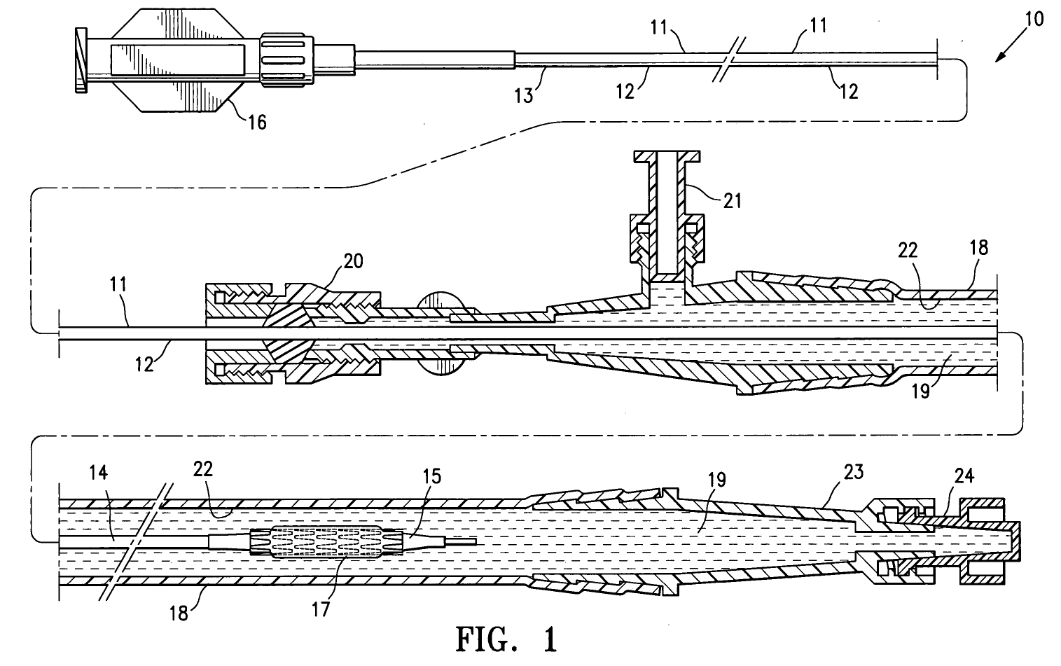

FIG. 1 illustrates a protected stent delivery system 10 embodying features of the invention. The system 10 includes a delivery catheter 11 which has an elongated shaft 12 with a relatively stiff proximal shaft section 13, a flexible distal shaft section 14, an inflatable balloon 15 on the distal section and an adapter 16 mounted on the proximal end of the elongated shaft. An expandable stent 17 is mounted on the working section of the balloon 15. A protective sheath 18 is disposed about the distal shaft section 14. The protective sheath 18 is filled with a body of protective fluid 19 which minimizes detrimental affects to the stent 18 and allows the stent to be used by the physician upon removal from the package and possible rinsing the distal shaft section and mounted balloon before inserting into the patient. The proximal end of the sheath 18 has a hemostatic valve 20 configured to sealingly engage the exterior of catheter shaft 12. A side port 21 is also provided on the proximal end of the sheath 18 to allow for the introduction of fluid into and the withdrawal of fluids from the interior 22 of the sheath 18 to remove protective fluid and rinse the distal shaft section with an appropriate solution prior to use. The distal end 23 of the protective sheath 18 is provided with an accessing port 24 which either alone or in conjunction with the side port 21 at the proximal end of the sheath 17 allows for the introduction of fluids into and the withdrawal of fluids from the interior of the sheath 18.

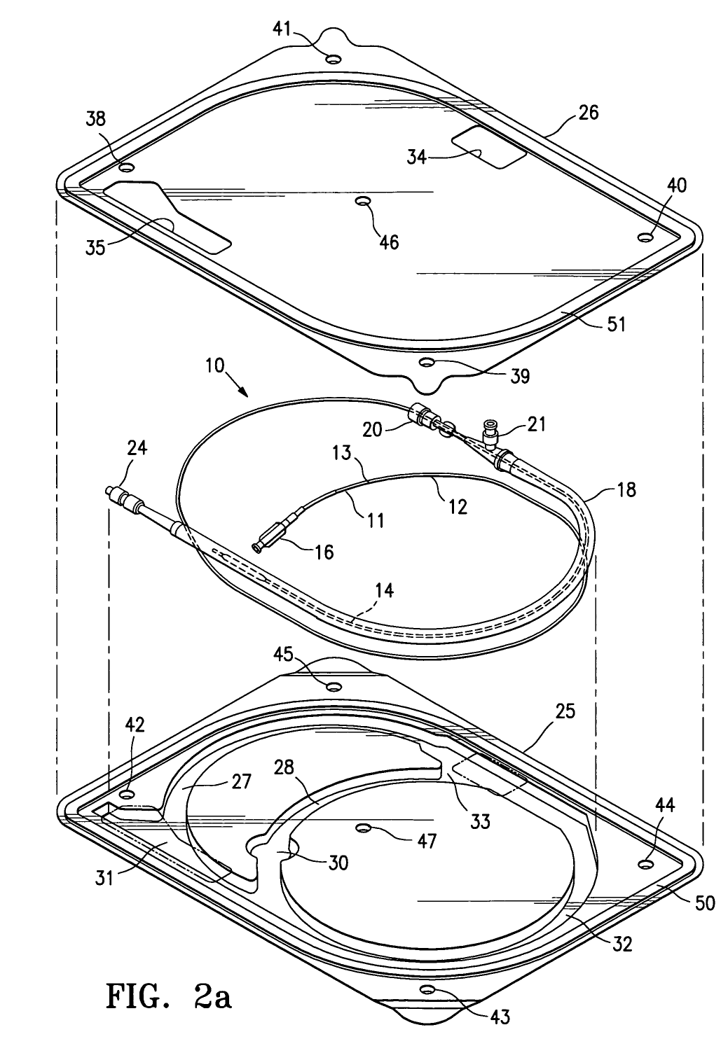

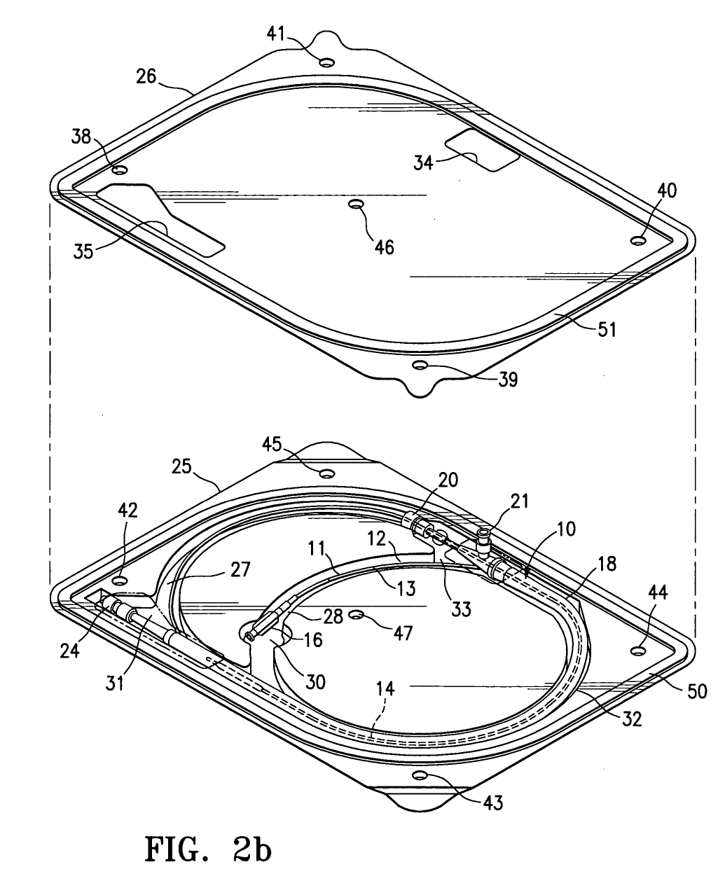

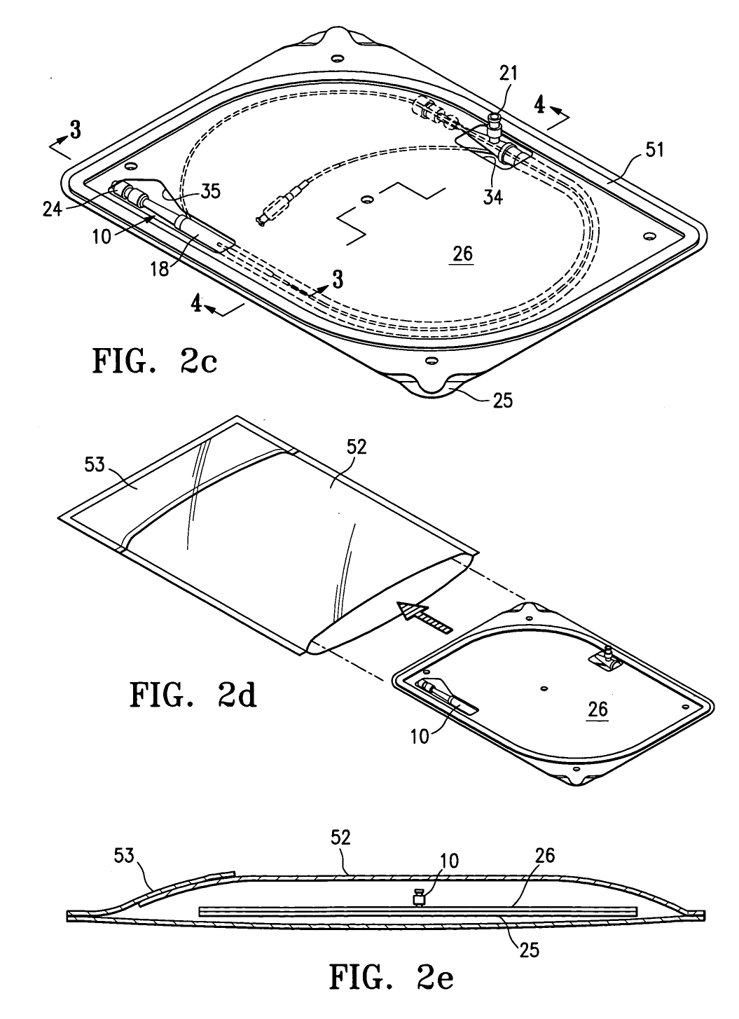

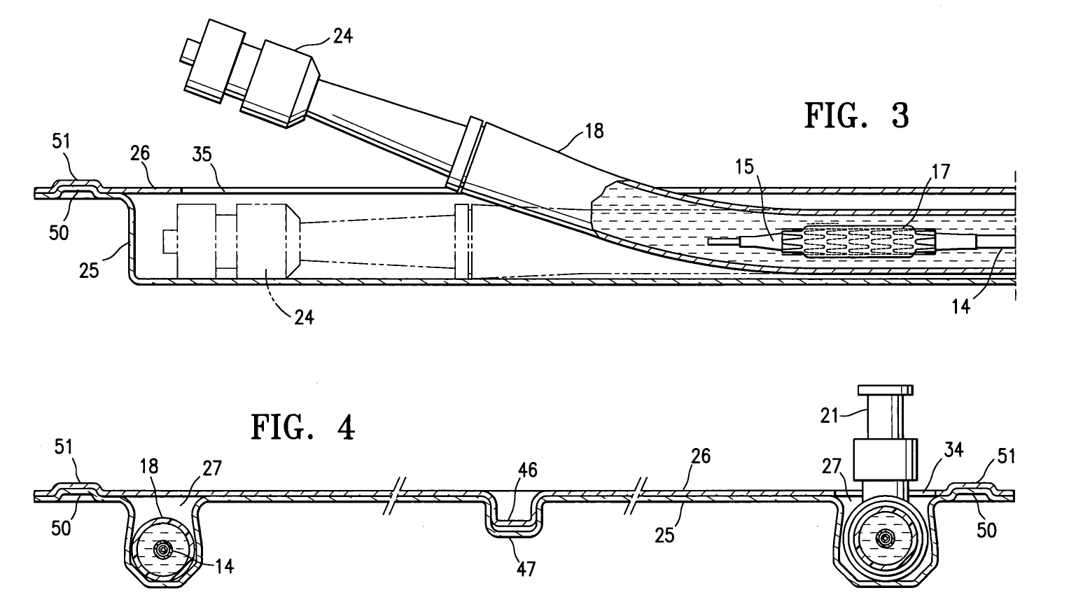

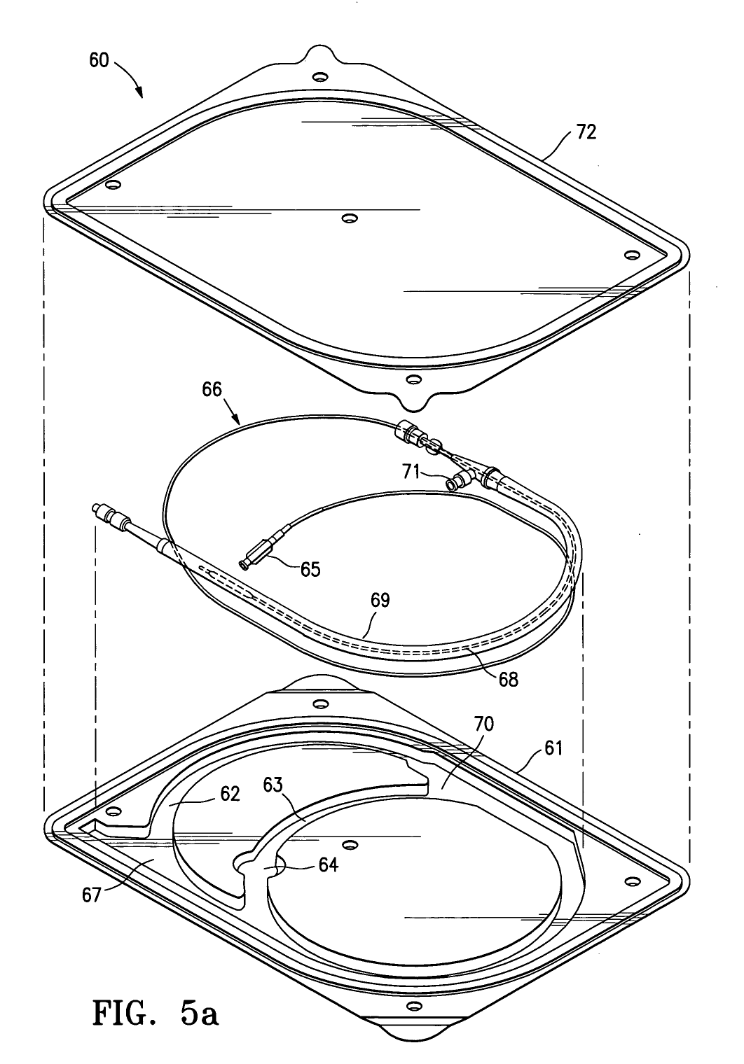

FIGS. 2a-2c illustrate an exploded view of a tray 25 and a tray cover suitable for the protected stent delivery system 10 with protective sheath 18 on the distal section 14. The upper surface of the tray 25 is provided with an oval recess 27 and an arcuate cross-over recess 28 configured to receive the stent delivery system 10. A well 30 is provided to receive the adaptor 16 at the proximal end of catheter shaft 12 and has larger dimensions that the crossover recess 28 to allow operating personnel to easily pick up the system 10 by the adapter 16. A run-out well 31 is provided to receive the distal end of the protective sheath 18. The sheath 18 extends from the run-out well 31 in the oval recess 27 around the curved end 32 thereof to the upper junction 33 between oval recess 27 and the cross-over recess 28. The tray cover 26 has a first opening 34 to allow the side port 21 (as shown in FIGS. 2c and 4) to extend therethrough. Similarly, the tray cover 26 has a second opening 35 to allow the distal end of the sheath 18 to be pulled through the opening to allow access to the accessing port 24 as shown in FIG. 3. This construction allows for the addition to or the withdrawal of fluids from the interior of the protective sheath 18, while the protected stent delivery system 10 remains protected in the covered tray. The corners of the tray cover 26 have projections 38-41 which fit into the corresponding recesses 42-45 at the corners of the tray 25 to provide a snap fit between the tray 25 and the cover 26. A centrally located projection 46 is provided in the cover 26 and a matching centrally location recess 47 is provided in the tray 25. A sealing ridge 50 is provided around the periphery of the upper surface of tray 25 and a matching sealing recess 51 is provided around the periphery of cover 26. As shown more clearly in FIG. 4 the recess 27 (and also recess 28 which is not shown) is provided with upper slightly overhanging lips to hold the stent delivery system in the recess during handling, transporting and storage while allowing its easy removal.

With protected stent delivery system disposed on the tray 25 and cover 26 in place, the covered tray may then be placed within a sealable envelope or pouch 52 as shown in FIG. 2d. Once the covered tray with stent delivery system 10 is placed within the interior of pouch 52, the lower edges of pouch 52 are sealed by heat or adhesive after the covered tray is inserted into the pouch. The securing flap 53 is presealed to the exterior of the pouch as shown in FIG. 2e and is configured to be used by the physician to open the pouch. At least part of the pouch 52 is formed of a permeable material such as Tyvek which is permeable to a sterilizing gas such as (EtO).

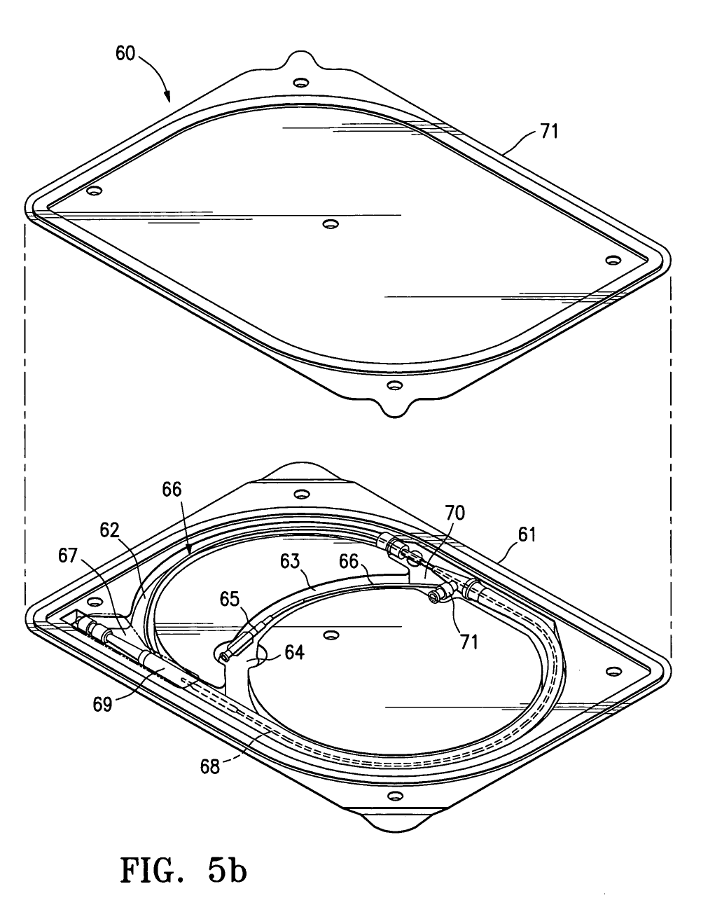

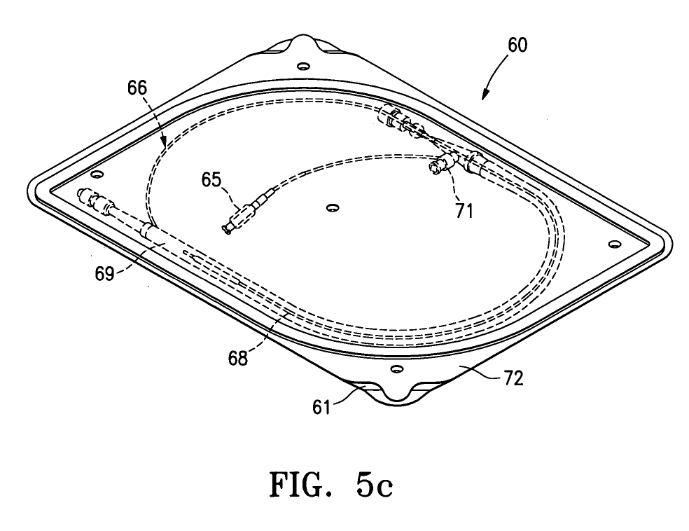

FIGS. 5a-5c illustrate an alternative sterilizable package system 60. The package system 60 has a tray 61 as described in the previous embodiment with an oval recess 62, an arcuate cross-over recess 63, circular well 64 configured to receive the adapter 65 from the delivery catheter 66 and run-out well 67 for the distal shaft section 68 with protective sheath 69. The tray 61 has an additional recess 70 configured to receive the side port 71 on the proximal portion of the sheath 69. The tray cover 72 is essentially the same as the tray cover described in the previously discussed embodiment except that it has no openings. This allows the tray and cover to be sealed and then sterilized as in the previous embodiment without the need for a pouch or other additional container. The tray cover may be formed at least in part of permeable materials.

Details of the stent and a stent jacket are described in U.S. Pat. No. 6,605,107, and a suitable stent delivery catheter is described in co-pending application Ser. No. 10/735,548.

The stent delivery catheter may be provided in either a rapid exchange design which has a proximal guide wire port a short distance, e.g. 5 to about 50, preferably about 10 to about 40 cm, from the distal end of the catheter or an over-the-wire design as described above in which the guide wire lumen extends the length of the catheter and is in fluid communication with the port in the proximal end of the catheter.

The catheter components may be formed of conventional materials used in angioplasty and stent delivery catheters and may be formed in a conventional manner. The elongated catheter shaft will generally have the dimensions of conventional dilatation or stent delivery catheters. The length of the catheter, measured from the distal end of the adapter 16 to the distal end of the elongated catheter shaft 11 may be about 90 cm to about 150 cm, and typically, it is about 137 cm.

The tubular protective sheath may be formed of suitable polymeric materials such as Tygon® or C-Flex® tubing and is configured to accommodate the distal extremity of the delivery catheter and a mounted stent.

The gas permeable portion of the container will typically be composed of a material which is permeable to the sterilizing gas and impermeable to liquids. Suitable gas permeable materials include high-density polyethylene (HDPE), typically a spun HDPE or other spun olefin. Commercially available materials such as Tyvek® (2FS, 1059B and 1073B) are specifically designed for packaging medical devices. Typically, the available materials are coated partially or totally with an adhesive for the purpose of sealing the material to itself or to adjacent materials.

The permeability required for the sterilizing gas to permeate into the interior of the container and contact the one or more articles therein, can vary depending upon the pressure differential, the temperature and the time available for permeation of the sterilizing gas into the interior.

The container may have both a gas permeable portion, as described above, and a gas impermeable portion, typically formed from a metal or polymer film which is impermeable to gases. Exemplary suitable material for the gas impermeable portion include metal foils such as aluminum, polymer films such as polyethylene, polyester, polyester (PET)/modified low density polyethylene (LDPE) laminated film, or a laminate of the two. (i.e., metalized PET).

The permeable portion or portions of the container may be incorporated with suitable oxygen scavenger materials or may be positioned adjacent to a partial or complete film of suitable oxygen scavengers. Not only oxygen is removed from the package, oxygen gas trying to permeate through the permeable portion will be limited from entering the package.

Oxygen scavengers can be placed in fluid communication with the interior of the package, such as within a permeable packet that is placed within the interior of the container or within the interior of an enclosure surrounding the container. Oxygen scavengers may be embedded in the material of the container or in a pouch which receives the container.

While particular forms of the invention have been illustrated and described, it will be apparent that various modifications and improvements can be made to the invention. For example, the stent may be coated or provided with a jacket having one or more therapeutic or diagnostic agent incorporated therein. Unless described otherwise, conventional materials and methods of construction may be used to make the catheters and stents. Although individual features of embodiments of the invention may be shown in some drawings and not in others, those skilled in the art will recognize that individual features of one embodiment of the invention can be combined with any or all the features of another embodiment. Accordingly, it is not intended that the invention be limited to the specific embodiments illustrated. It is therefore intended that this invention to be defined by the scope of the appended claims as broadly as the prior art will permit.

Terms such a “element”, “member”, “device”, “section”, “portion”, “step”, “means” and words of similar import when used herein shall not be construed as invoking the provisions of 35 U.S.C. §112(6) unless the following claims expressly use the term “means” followed by a particular function without specific structure or the term “step” followed by a particular function without specific action. All patents and patent applications referred to above are hereby incorporated by reference in their entirety.

1. A protected stent delivery system for a jacketed or coated stent, comprising:
a. a delivery catheter which has an elongated shaft, a distal extremity on the elongated shaft and a stent expanding member on the distal extremity of the catheter;
b. a jacketed or coated stent mounted on the stent expanding member; and
c. a sealable container disposed about the distal extremity of the catheter which has an interior configured to receive the expandable member and the stent mounted thereon and which is configured to maintain a body of protective fluid within the interior of the container.

2. The delivery system of claim 1 wherein the sealable container is a sheath which has an inner lumen.

3. The delivery system of claim 2 wherein the sheath has a proximal end and a hemostatic closure at the proximal end.

4. The delivery system of claim 3 wherein the sheath has a distal end and a first accessing valve at the distal end.

5. The delivery system of claim 2 wherein the sheath is a tubular member.

6. The delivery system of claim 3 wherein the hemostatic closure includes a second accessing valve which provides access to the inner lumen of the sheath.

7. The delivery system of claim 3 wherein the hemostatic closure is configured to sealingly engage the elongated shaft of the catheter proximal to the expandable member.

8. The delivery system of claim 1 wherein the distal end of the sealable container extends distal to the delivery catheter.

9. The delivery system of claim 1 wherein the interior of the sealable container is at least partially filled with protective fluid.

10. The delivery system of claim 1 wherein the stent has a jacket formed at least in part of heterologous tissue.

11. The delivery catheter of claim 10 wherein the heterologous tissue is selected from the group consisting of porcine pericardial tissue, bovine pericardial tissue and equine pericardial tissue.

12. The delivery catheter of claim 10 wherein a therapeutic or diagnostic agent has been incorporated into the heterologous tissue.

13. The delivery catheter of claim 12 wherein a therapeutic agent selected from the group consisting of rapamysin and paclitaxel is incorporated into the heterologous tissue.

14. The delivery catheter of claim 1 wherein the stent has a coating into which a therapeutic or diagnostic agent has been incorporated.

15. The delivery catheter of claim 14 wherein a therapeutic agent selected from the group consisting of rapamysin and paclitaxel is incorporated into the stent coating.

16. The delivery catheter of claim 14 wherein the coating is biodegradable.

17. A sterilized package containing a stent mounted on an inflatable member of a delivery catheter, comprising:
a. a support tray configured to receive an elongated delivery catheter with a proximal end and a distal end, an inflatable member proximally spaced from the distal end, an expandable stent mounted on the inflatable member and a sheath which has an inner lumen configured to receive the inflatable member of the delivery catheter and the stent mounted thereon and which is configured to maintain a protective fluid within the inner lumen in contact with the stent; and
b. a tray covering which defines in part a package interior and which is configured to retain sterile conditions within the package interior.

18. The package of claim 17 wherein at least part of the tray or tray covering is formed of material which is permeable to a sterilizing gas.

19. The package of claim 17 wherein the sheath is configured to sealingly receive the inflatable member and stent mounted thereon within the inner lumen of the tubular member.

20. The package of claim 17 wherein the sheath is disposed within the interior of the package.

21. The package of claim 20 wherein the sheath has at least one sealable port which facilitates introduction or withdrawal of fluid into the inner lumen of the tubular member.

22. The package of claim 21 wherein the sheath has a sealable port at one end thereof and a sealable port at another end thereof.

23. The package of claim 21 wherein the sheath has at least one port accessible from an exterior location of the package to allow fluid introduction or withdrawal from outside the package without interfering with the sterile conditions within the interior of the package.

24. The package of claim 17 wherein the tray has a recess for receiving at least part of the delivery catheter and mounted stent.

25. The package of claim 18 wherein at least part of the tray or the tray cover is formed of a permeable spun polyolefin.

26. The package of claim 25 wherein the spun polyolefin is a high density poly-ethylene.

27. The package of claim 26 wherein the spun high density poly-ethylene is permeable to EtO.

28. The package of claim 18 wherein the tray and tray cover are sealably secured together about the margins thereof.

29. A sterilized package, comprising:
a. a sealed container which has an inner chamber defined at least in part by a wall, which has at least a portion permeable to sterilizing gas and which has at least one sterilized article within the inner chamber; and
b. a sealed sheath which is disposed about at least a portion of the sterilized article and which has a protective fluid within an interior thereof.

30. The sterilized package of claim 29 wherein the at least one article is a stent.

31. The sterilized package of claim 30 wherein the stent has a jacket formed at least in part of heterologous tissue.

32. The sterilized package of claim 30 wherein the stent is a drug eluting stent.

33. The sterilized package of claim 32 wherein the drug eluting stent has a polymeric coating with an eluting drug incorporated therein.

34. The sterilized package of claim 33 wherein the polymeric coating is bioresorbable.

35. The sterilized package of claim 31 wherein the drug eluting stent has a jacket of pericardial tissue with an eluting drug incorporated therein.

36. The sterilized package of claim 30 wherein the stent is mounted on a balloon of a delivery catheter.

37. The sterilized package of claim 29 wherein an oxygen scavenger is maintained in fluid communication with the inner chamber of the container.

38. The sterilized package of claim 29 wherein the container includes a tray and a cover.

39. The sterilized package of claim 38 wherein the tray and a tray cover are disposed within a pouch.

40. The sterilized package of claim 39 wherein the pouch is formed at least in part of a material which is permeable to sterilizing gas.

41. A method of packaging and sterilizing a medical device, comprising:
a. providing a container which has an inner chamber defined at least in part by a wall and which has at least a wall portion permeable to sterilizing gas;
b. sterilizing at least a portion of a medical device;
c. surrounding at least a sterilized portion of the medical device with a sealable sheath having an interior configured to receive a body of fluid to protect the sterilized portion of the medical device disposed therein;
d. disposing the at least partially sterilized medical device and sheath within the inner chamber of the container; and
e. contacting an exterior part of the permeable portion of the container with sterilizing gas under conditions which cause the sterilizing gas to penetrate the permeable portion to sterilize at least a portion of the medical device within the inner chamber.

42. The method of claim 41 wherein the medical device is a delivery catheter with an inflatable balloon and a drug eluting stent mounted on the balloon.

43. The method of claim 41 wherein the sterilizing gas is ethylene oxide.

44. A sterilized packaging system for a medical device or component, comprising:
a. a support tray configured to receive a medical device or component, a sheath which has an interior which retains at least part of the medical device or component and which is configured to maintain a protective fluid within the interior in contact with the device or component; and
b. a tray covering which is sealingly secured about the margin thereof to the tray, which defines in part a package interior containing the medical device or component and the sheath and which is configured to retain sterile conditions within the package interior;
c. at least part of the tray or tray covering formed of a material which is permeable to sterilizing gas but is impermeable to pathogens.
